(19) United States
(12) Patent Application Publication
(10) Pub. No.: US 2021/0275400 A1
(43) Pub. Date: Sep. 9, 2021

(54) Apparatuses for use with Replacement Gastrostomy Tubes for Status Indication, Durability and Design of Temporary Distal Tips

(71) Applicant: Ethan Hiram Dubin, Baltimore, MD (US)

(72) Inventor: Ethan Hiram Dubin, Baltimore, MD (US)

(21) Appl. No.: 16/807,693

(22) Filed: Mar. 3, 2020

(51) Int. Cl. A61J 15/00; A61B 17/34

(52) U.S. Cl. CPC A61J 15/0015; A61J 15/0049; A61M 25/0069; A61B 17/3468; A61J 15/0088

(57) ABSTRACT

Assembly of devices, and a kit, for use in replacement gastrostomy tube therapy, consisting of: 1) a Replacement Gastrostomy Tube status device comprising an evaluation module able to trigger an alarm that indicates the inflation status of a retention balloon; 2) a temporary distal “tip” structure for use in centering the Replacement Gastrostomy Tubes in vivo; and 3) a gastrostomy tube comprising a mesh latticework structure to increase the structural integrity of the Replacement Gastrostomy Tubes; and/or a pH sensor measuring acidity of fluid at distal tube opening. The module/unit comprises a battery powered, visual light, auditory signal or network-connected communication to convey the retention balloon's inflation status. The temporary distal “tip” structure includes various tapers, with minimal distal area, to facilitate placement of the Replacement Gastrostomy Tube via a self-centering function, in which the tip is chemically or mechanically released into a digestive tract, or withdrawn externally after a Replacement Gastrostomy Tube is positioned in vivo.

### FIELD OF THE INVENTION

The present invention relates to improvements in status monitoring of in-dwelling retention balloons for Replacement Gastrostomy Tube feeding devices, placement of such tubes, and improved structural rigidity of such tubes. More particularly, the present invention relates to the overall construction and configuration of gastrostomy tubes and/or enteral feeding catheters having a portion which is deployed outside a living body, and a tube which is inserted through a stoma to a point inside the body for ongoing delivery of nutrition and medications.

### BACKGROUND OF THE INVENTION

Numerous inventions exist in the prior art which create the function of a gastrostomy tube for the purpose of delivering nutritional substances or other compounds directly to a patient's digestive tract within a living body. A subset of these devices are considered replacement gastrostomy tubes and they specifically perform the function of delivering nutritional substances through a surgically created opening to a point typically inside a patient's digestive tract, most commonly inside a patient's stomach. It is particularly important for medical practitioners inserting these devices to confirm placement inside the stomach and to facilitate rapid, accurate placement when replacement is required, and to maximize the lifespan of such tubes thereby minimizing the need for such tubes to be replaced.

Initially, Gastrostomy tubes are surgically placed (Percutaneous Endoscopically placed Gastrostomy Tubes—PEG). These eventually require replacement with a replacement gastrostomy tube. Replacement gastrostomy tubes wear out quickly and several replacements may be necessary per year due to, for example, deformation of a tube and build-up of pathogens on a tube. Additionally, such tubes are anchored within a patient's stomach by a balloon-like structure which may slowly spontaneously deflate. This may cause a tube to fall out and a visit to an emergency room may be required in order to replace a tube. Mechanisms and methods to detect deflation occurring before a tube may become dislodged are not provided by most currently manufactured tube designs, and advances in the related ability offer the potential for improvement in patient care. Mechanisms and methods to improve communication and detection of inflation status of an internal balloon are disclosed in the present invention as well as improvements to facilitate and confirm correct placement of said tubes. Additionally, a method to increase the structural integrity of such tubes is disclosed.

Existing devices have several shortcomings that create negative consequences for patients and caregivers. Existing Replacement Gastrostomy Tubes as customarily designed have a “balloon-like” structure internal to the patient's body after the tube is placed in positon for its function. The balloon structure is inflated with a fluid and is a primary mechanism of retaining a tube at a necessary in-dwelling position. A common failure for such tubes is loss of fluid from the retention balloon over time which creates a risk that a tube will become dislodged when the retaining action of the balloon is lost due to loss of fluid and consequent loss of shape in the in-dwelling balloon structure. Replacement fluid can be added via an external filling port to restore the retention ability of the in-dwelling balloon, however a need for additional fluid for an internal retention balloon is not easily or readily apparent in many prior art designs, or is not of a relatively simple, inexpensive and reliable design. The present invention seeks to resolve these deficiencies by disclosing methods and mechanisms for detecting and communicating the inflation status of such internal retention balloons in simple, reliable and broadly detectable manners.

Existing devices and other information disclosed in prior art lack several key advances included in the present invention. Extensive prior art addresses the broad construction of gastrostomy tubes. A disclosure which relates to similar topics as the present invention is included in patent U.S. Pat. No. 8,177,742 B1 which discloses a method and mechanism for monitoring the inflation status of the internal retention balloon by means of a visual mechanical indicator triggered by a pre-biased inflation pressure sensing mechanism, among other disclosures. The mechanism disclosed however, as will be described, has significant operational and manufacturing complexity in comparison to the present invention, and does not disclose means of communicating the inflation status beyond basic visual means.

U.S. Pat. No. 6,732,734 discloses an external “pilot” balloon that also provides a form of visual and tactile status indication, however the method of connection to the in-dwelling retention balloon requires an air tube, and the reliance on a visual signal which may not occur until significant loss of inflation pressure in the in-dwelling retention balloon. The loss of pressure in in-dwelling retention balloons remain a significant problem for practitioners using this device. These short-comings create the need for a signal mechanism which offers simpler implementation, manufacturing simplicity and a broader means of status communication for inflation status of the in-dwelling retention balloons.

Additionally, moving beyond retention balloon inflation status, placing replacement gastrostomy tubes in their in-dwelling, ongoing operational position requires a medical specialist to insert a replacement gastrostomy tube through an opening in a portion of a digestive tract. Such an opening is not visible to a practitioner and such situations can create difficulty, risk and unproductive time spent aligning a tube with such an opening. Additionally, the body opening through which a tube passes begins to close rapidly after a tube is withdrawn, further increasing the risk and difficulty in positioning a replacement tube. Prior art attempts to partially address these shortcomings by disclosing forms and degrees of taper in the distal tip of a replacement gastrostomy tube such that the tip is smaller than the circumference found in a predominant length of such a tube. The diameter of existing replacement gastrostomy tubes is often, gradually reduced as the tube approaches its distal opening which forms a “tapered” shape which creates a degree of “self-centering” action as such a tube is inserted into a surgically created opening., The degree of taper in such tubes is meaningfully limited however, because a distal opening must remain with a sufficiently large area and shape such as to allow passage of a sufficient quantity and type of nutrient and medication necessary to support the purpose of such gastrostomy tubes. To address the preceding deficiency, a portion of the present invention discloses design and construction principles for a form of temporary distal tip which is designed to remain in place during insertion of a tube and then be removed via some combination of one or more actions including dissolution of the temporary tip by fluids commonly found in a digestive tract, deposition of the tip in a digestive tract by mechanical means of ejection or by the force of initial substances delivered through such a tube, and/or mechanical removal of the tip via withdrawal though the predominant length of a tube in a direction opposite a distal opening of a tube. The design principles for the temporary distal tip of the present invention create an ability for much greater taper than available in prior art, and create a distal tip with the potential for the smallest possible distal surface area, thus creating much-improved self-centering functionality to support placement of a tube in comparison to prior art while retaining the area of distal opening necessary for function of the tube in delivering nutrients and medications.

In some cases, it is possible to insert the replacement tube through the prior tract which had led to the stomach but instead, with insertion of the new tube, the new tube creates a break in the stoma allowing the tube to enter the peritoneal cavity such that a distal opening of a replacement gastrostomy tube assumes an in-dwelling location such that said distal opening will discharge feeding materials into an unintended location. Such a situation creates extreme danger and must be rapidly corrected, however technical means of confirming with high confidence that a tube distally terminates in a stomach is often not available to medical practitioners charged with inserting such tubes. Fluids uniquely present within a stomach are, in many cases, uniquely acidic within a digestive tract, and as such, confirmation that the distal tip of said replacement gastrostomy tubes is in contact with a highly acidic fluid provides a meaningful additional confirmation that a recently-placed tube's distal opening is in fact within a stomach. The present invention discloses a feature which, when incorporated into gastrostomy tubes of customary design allows methods to be employed to assess acidity of fluids drawn into a distal end of a gastrostomy tube, facilitating said assessment of a tube's in-dwelling placement location. For example, when sufficient fluid which has accumulated in the stomach and is then suctioned up into the tube via a syringe attached to the proximal port, contact between this fluid and a ph sensitive substance embedded within the tube of the present invention can indicate whether the acidity of the fluid is such as to confirm that the tube is in fact placed in a stomach and not misplaced in another cavity. In practice, this procedure of assessing whether such a tube is placed in a stomach via analysis of liquid drawn into such a tube will usually require that a fluid such as sterile water be injected through the tube in order to provide sufficient fluid in a stomach to withdraw and assess for ph level.

A further weakness of existing replacement gastrostomy tubes is a common tendency for the tubes to deform over time after being placed in a body, and the deformation can make the tubes collapse or deform to a shape that prevents delivery of nutrients and make the tubes no longer fit for purpose, thus mandating their replacement. This deformation is due to deficiencies in materials commonly used for the tubes in dimensions necessary to balance nutrient delivery and minimally invasive stoma dimensions. The present invention discloses a structural approach to improving the structural integrity, and hence longevity, of a tube while retaining tube dimensions similar to current replacement gastrostomy tubes.

### SUMMARY OF THE INVENTION

The present invention discloses methods and mechanisms to communicate inflation status of the in-dwelling balloon by additional means beyond a visual status gauge as disclosed in prior art. These means include wired or wireless communication to a remote location, triggering of a status communication device using a form of pressure-sensing mechanism in fluid contact with an in-dwelling retention balloon, and additional forms of visual and tactile status alerts such as a blinking light or auditory signal. Additionally, the present invention discloses a method of generating an alarm based on a particular status being detected, the alarm being communicated in any form whereby an external signal is generated based on a particular inflation status being detected, typically indicating reduced inflation and associated risk of undesired movement in the overall replacement gastrostomy tube device and associated risk of a tube falling out of a stomach.

To address the issues associated with previously disclosed methods as described herein, the present invention discloses a method of communicating the status of an in-dwelling retention balloon customarily found as part of a replacement gastrostomy tube. The method integrates output from any form of pressure-sensing device in fluid communication with an in-dwelling retention balloon, and communicates detected pressure information as a means of indicating the inflation status of the in-dwelling retention balloon. The pressure information may trigger local or remote alarms, using auditory, visual or remote network facilitated indicators of detection of a particular pressure (and corresponding inflation status) of an in-dwelling retention balloon. The communication may indicate a range of pressures and statuses, or trigger a communication based on detection of an adjustable, user-entered pressure to create an alarm function. The communication may also be remote via, wired, wireless, network-connected, or other means of remote communication. A power storage battery source may be included with the mechanism to power an alarm. Example alarms may include a light signal, such as a blinking light, an auditory signal such as an intermittent sound, and/or a remotely communicated alarm via wired or wireless network communications (or a combination thereof) to alert medical staff of low pressure in an internal retention balloon.

Additionally, the present invention discloses principles for designs and construction materials to create a temporary distal tip with a purpose of facilitating accurate and easy placement for replacement gastrostomy tubes with the tip having much greater self-centering taper than disclosed in prior art. As discussed herein, a key design challenge for existing replacement gastrostomy tubes is how to make the tube as easy to place in an opening within the digestive tract as possible. Existing designs attempt to address this need by incorporating varying degrees of taper between the permanent integrated distal opening and the remainder of the tube, however the degree of taper available in the prior art is limited by the need to retain a distal opening sufficiently large for nutrients to pass through with sufficient speed and quantity. Those familiar with the field and art will recognize that, for tubes meant for adults, this opening is customarily less than a diameter of 20 French for adult-use tubes which is created by a taper from the diameter from the remainder of such tubes. Tubes for pediatric use are typically smaller diameters. The present invention provides design and construction principles to create a temporary distal tip to be used during the placement into a stomach for replacement gastrostomy tubes of any dimensions and is not restricted to the diameter described above which are provided for background and context. The tip of the present invention, due to its temporary nature, is not constrained by the need for a size of distal opening to allow food to exit such tubes. The present invention accomplishes this by disclosing tip design principles for a temporary distal tip which includes taper to, for example, the point where minimal distal surface is retained, or any size of distal tip surface and shape which terminates in a diameter smaller than the opening size for the distal end of a replacement gastrostomy tube. This is accomplished by constructing the tip using materials which may dissolve and/or be removed from the device once it is placed in its in-dwelling position and which materials may enter a digestive tract or be withdrawn from such tubes. The tip device of the present invention may be dislodged from a tube by mechanical or chemical means once in place, or be forced out by food flow through such tubes, but in any case, the temporary tip of the present invention is removed for a tube to reveal an ongoing tip of necessary size and shape after a tube has been placed in position for ongoing use.

Additionally, to address the shortcomings in the previously known art related to deformation of a replacement gastrostomy tube due to weakening of the material of its construction over time, the present invention discloses a design and construction feature which reduces the potential for replacement tubes of customary design to deform with age. This feature consists of a reinforcement layer of materials suitable to achieve greater shape retention than customary unreinforced tubes using a uniform material, The present invention discloses incorporation of a latticework mesh structure incorporated into the construction material of replacement gastrostomy tubes and extending over some or all of the length of such tubes with intent to preserve a generally tubular shape, sufficient to allow such tubes to continue to perform their intended function, on both the internal lumen of such tubes and external circumference of such tubes. The reinforced portions of a main feeding tube and/or ancillary ports may extend over all, or a subset, of such tubes and ports.

Additionally, to address the shortcomings in prior art related to the potential for a replacement gastrostomy tube to be misplaced such that the distal opening of a tube is placed in a location other than a stomach, the present invention discloses a feature which, in combination with methods known to medical practitioners skilled in the related art, creates an enhanced means of confirming and documenting that distal openings of such tubes are located within a stomach. This feature embeds a ph-sensitive material in a location on the internal lumen of such tubes with the material being visible through the translucent nature of the tubes, and located within the portion of the tubes that will be external to the body-surface stoma and visible to a medical practitioner when the tubes are placed in their in-dwelling position. This ph-sensitive material will provide, when fluid at the distal opening of an in-dwelling tube is withdrawn and placed in contact with the previously described ph-sensitive material, both an indication, based on the ph-of the fluid as indicated by visual signs from the ph-sensitive material, of whether stomach placement of a distal opening can be confirmed.

### BRIEF DESCRIPTION OF THE DRAWINGS

The included drawings depict the present invention integrated with a replacement Gastrostomy tube of customary design, as well as the distinct components of the present invention.

FIG. 1A is a side view of an example structure for the status device for a feeding tube internal retention balloon

FIG. 1B is a perspective view of an example structure for the status device for a feeding tube internal retention balloon

FIG. 1C shows a basic block diagram of example functional flows for the inflation status device.

FIG. 2 illustrates an exemplary embodiment of the status device in position on a standard replacement gastrostomy feeding tube and positioned within a body.

FIG. 2 also depicts an example of the anatomical positioning of a replacement gastrostomy tube including the elements of the external status device portion of the present invention in relation to a patient's body with delineation between specific in-dwelling elements and external elements when a tube has been positioned for feeding at a gastrostomy site.

FIG. 3 depicts an example design of the temporary tip portion of the present invention placed within the distal opening of a replacement gastrostomy tube as it would be placed to facilitate placement of such a tube.

FIG. 4 depicts an example design of the temporary tip portion of the present invention in the context of using a rod-and-swab type of mechanical device to dislodge the tip into a digestive tract. A similar path of dislodgement would occur if food or other substance passing through the feeding tube dislodges the tip of the present invention.

FIG. 5 depicts an example design of the temporary tip portion of the present invention in the context of using a mechanical attachment and elongated substance such as a string or rod to apply extractive force to the tip such that the tip is withdrawn through an external opening in a gastrostomy tube.

FIG. 6A depicts a cross-sectional view of a gastrostomy tube with a mesh-latticework reinforcement which may be embedded within some or all of the length of a feeding tube and in some or all of the adjunct tube extensions/ports as a form of structural reinforcement to increase the functional life of a tube by reducing the potential for a tube to deform from its initially constructed shape.

FIG. 6B is a side view of FIG. 6A with the outer tube cut away to illustrate the mesh-latticework reinforcement.

FIG. 7A is an axial cross-sectional view of a gastrostomy tube with a ph-sensitive material embedded within the interior surface of a replacement gastrostomy tube such that the material will register an approximation of the ph of fluid withdrawn from the location of the in-dwelling distal tip deposited on the material, which will be visible to a medical practitioner and will have precision sufficient to assess if the distal tip is in contact with stomach acid.

FIG. 7B is a longitudinal cross-sectional view of the gastrostomy tube of FIG. 7A, illustrating the pH sensitive material in a top-plan view.

### DETAILED DESCRIPTION OF EXEMPLARY EMBODIMENTS

Reference will now be made in detail to one or more embodiments, examples of which are illustrated in the drawings. It should be understood that features illustrated or described as part of one embodiment may be used with another embodiment to yield still a further embodiment. The present invention comprises an assembly for use in Replacement Gastrostomy Tubes, comprising one or more of: a status device able to trigger an alarm that indicates the inflation status of a retention balloon; 2) a temporary distal “tip” structure for use in centering the replacement gastrostomy tubes in vivo; 3) a gastrostomy tube design comprising an inner mesh latticework structure to increase the structural integrity of such tubes; and 4) a gastrostomy tube design enhancement comprising a pH sensor for measuring acidity of fluid at a distal tube opening.

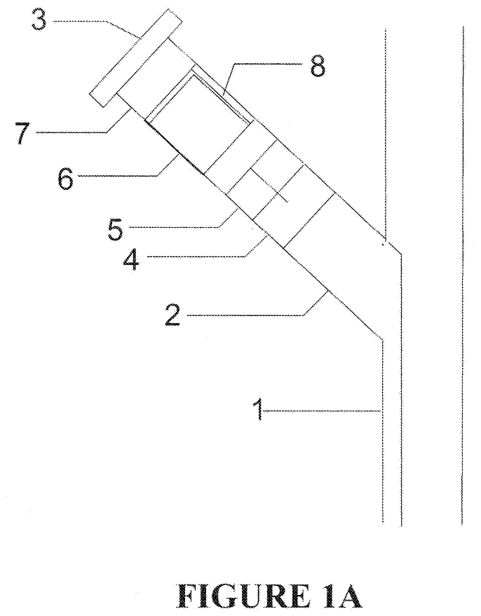

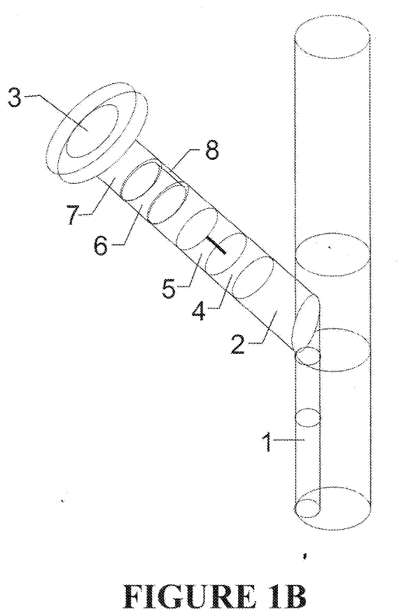

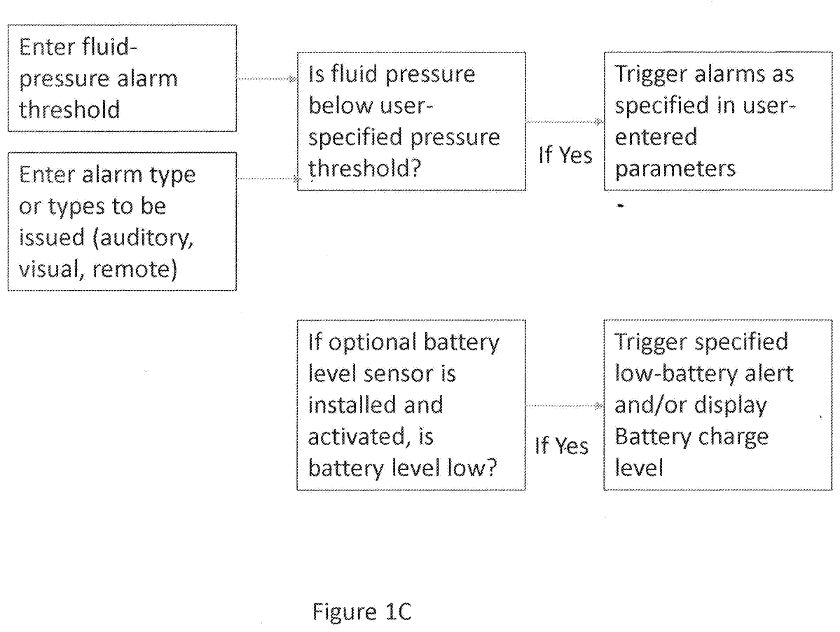

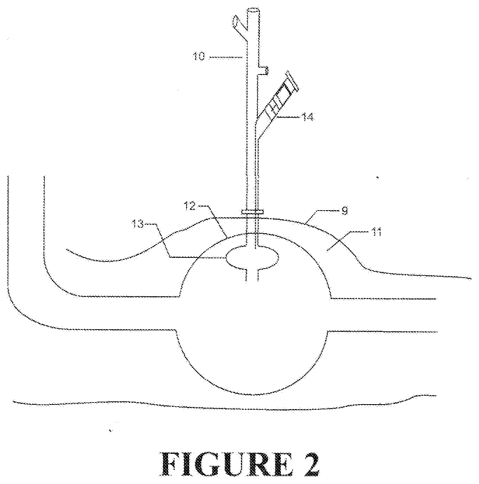

An exemplary embodiment of the status device 13 is illustrated in FIGS. 1-2, wherein the status device 14 comprises: a battery powered, visual light, auditory signal or network-connected communication to convey the balloon's inflation status. Note that while FIG. 2 depicts a straight tube exiting at a 90 degree angle from the surface of a body, any shape of tube is appropriate for the present invention and the depiction is simply to provide a general reference. Most such tubes can be expected to bend approximately 90 degrees as they exit a body in order to conform to a body position.

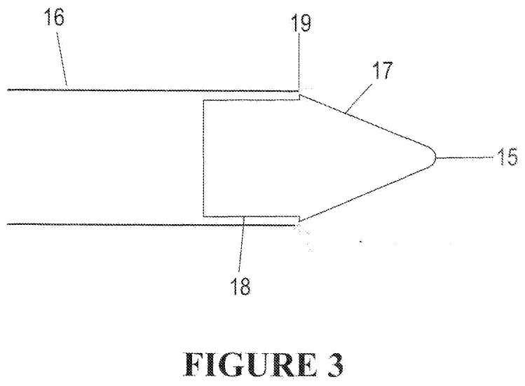

Various exemplary embodiments of the temporary distal tip are illustrated in FIGS. 3-5. The temporary distal “tip” structure comprises various taper angles, with minimal distal area, to facilitate placement of the replacement gastrostomy tube via a self-centering function, in which the tip is chemically or mechanically released into a digestive tract, or withdrawn externally after a replacement gastrostomy tube is positioned in vivo.

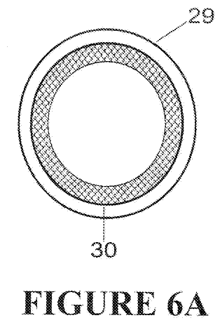

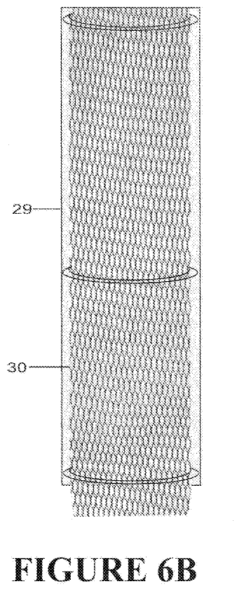

FIGS. 6A and 6B are an exemplary embodiment of a gastrostomy tube comprising an inner mesh latticework structure of the present invention that functions to increase the structural integrity of such tubes.

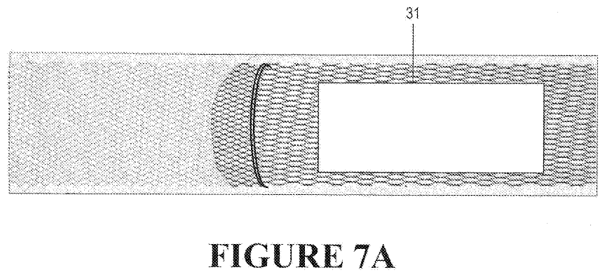

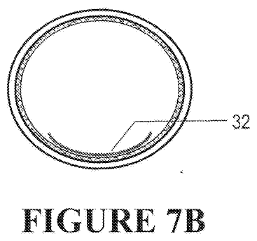

FIGS. 7A and 7B illustrate a gastrostomy tube comprising a pH sensor strip affixed to the interior surface of the tube in order to measure the acidity of fluid at the distal tube opening. pH in the range of the stomach (which is typically in a range of 1.5 to 3.5), which indicates to a clinician that a tube is properly positioned in a stomach, as opposed to outside of a stomach.

In one or more embodiments, the present invention comprises a kit with written instructions, or a posted link to online instructions on use of the kit, and wherein the kit comprises one of more of the devices of FIGS. 1-7B.

### DETAILED DESCRIPTION OF THE INVENTION

### Internal Retention Balloon Status Device

Specifically focusing on FIGS. 1A and 1B, these figures depict an apparatus which could be fitted to a gastrostomy tube port 2, the port being in fluid communication with an internal retention balloon as commonly found on such devices. The apparatus of the presently disclosed invention is comprises a means of generating visual, auditory, and/or network-connected signals based on the inflation status of the internal retention balloon as detected by any form of pressure assessment of the communication fluid which indicates the inflation status of an internal retention balloon as commonly found on such tubes.

The disclosed apparatus may be configured to send or display communication signals indicating the pressure status in a stand-alone manner and/or to other devices, such alarms including but not limited to audible alarms with optionally configurable volume, network connected data and alarms, or other forms of wired or wireless data transport where the data indicates the status of the pressure detected from fluid connection to the internal retention balloon. The placement and design as depicted are shown as examples and the present invention extends to any form of incorporation of a pressure-sensing device in fluid communication with the in-dwelling retention balloon, and which includes a device to display, emit or convey a signal which may be interpreted as an indication of low pressure and consequently degraded inflation status of an internal retention balloon. A battery power source or other source of stored power may also be included to power the generation of the signal. This power source may be replaceable or permanently embedded within the device.

Addressing the specifics of FIGS. 1A and 1B, a generic example segment of a gastrostomy tube is depicted, the tube including a port 2 in fluid communication with an internal retention balloon by means of an embedded fluid-tight channel 1 within a replacement gastrostomy tube. The channel boundaries indicated 1 show an example of a fluid-tight channel 1, however any means of establishing fluid communication with an in-dwelling retention balloon is covered in the present invention. The channel is assumed to connect to a form of port 2 or other terminating end-point to which the apparatus of the present invention may interact with via any means of assessing pressure of the fluid. FIGS. 1A and 1B depict an example embodiment of such a port (2) for ease of depiction, a port being defined as any opening or access point to the interior channel of a feeding tube other than the distal opening.

The present invention includes a sensing unit module 4 comprising hardware device receiving input from any form of pressure sensing apparatus that detects when a pressure threshold is reached for the retention-balloon fluid that is in communication with an in-dwelling retention balloon. The device may produce, upon triggering of a low-pressure threshold, any combination of an auditory alarm via an alarm module 3 such as a “beeping” tone, a visual signal such as some form of light, or a data signal such as would be connected to a network, or wirelessly communicated to a separate signaling apparatus. Additionally, the invention may optionally allow a user to specify the type of alarm or alarms that are issued when a low-pressure situation is detected by the device.

Assessment of pressure in the communication fluid may be performed by any means disclosed in prior art as depicted by the sensing module 4 (e.g. a pressure sensor of prior art) and transmitted to the presently disclosed invention via an interface point 5. A location for a form of power storage 6 is depicted which powers circuitry 7 which serves to interpret data from the pressure sensing mechanism 4 and to trigger and control the alarm function module 3. The circuitry is in physical or wireless connection 8 with the sensor interface 5 and the alarm mechanism 3. The physical implementation including, for example the placement and sequence of the modules described may vary in any manner provided the overall functions of the device are achieved as specified herein. Additionally, the invention may optionally allow a user to specify the pressure at which an alarm is triggered. Examples of such adjustment mechanisms may include digital entry of a precise pressure below which would trigger an alarm or a simple relative pressure selection such a high/medium/low, however any form of pressure specification is within the scope of the present invention.

The depiction of FIGS. 1A and 1B and all figures in this application are examples of the conceptual components minimally required for the present invention and do not depict the precise physical, special or logical layout of an actual device implementing the presently disclosed capabilities. The current depiction is only for ease of conveying the concepts covered by the present invention and exact implementation details may vary as known to those familiar with the art associated with electronic devices.

FIG. 1C is a block diagram depicting an example of actions and flows possible with the currently disclosed invention. Specifically, a user may enter one or more specific pressures at or below which an alarm may be triggered. This could over-ride a pre-set threshold or be the only form of threshold depending on the discretion of the device manufacturer. A user may also select one or more alarm types to be issued at a given threshold or different alarms based on different pressure thresholds. For example, an auditory alarm could be triggered at an initial threshold of low pressure and a remote alarm could be triggered by an additional lower pressure indicating greater urgency. In additional to pressure sensing and alarm generation, the device may optionally also generate an indication of a low battery condition, however this is not a mandatory feature of the invention. Low battery status or general charge level of a battery may be indicated by one of the alarm functions as used for the pressure-sensing function, or by a separate alarm or display associated with the battery.

Turning to FIG. 2, the present invention depicted in FIGS. 1A and 1B is, in FIG. 2, depicted in the context of an example tube's 10 in-dwelling position when placed in its operational position within a body. The example replacement gastrostomy tube intersects the outermost boundary of the skin on a patient's body 9 and further intersects a peritoneal cavity 11. A conceptual stomach 12 is depicted for further context where a retention balloon 13 as depicted in prior art is located. The customary portions of a replacement gastrostomy tube 10, including both those external and internal to a body are conceptually depicted. The unique feature of the present invention described previously herein, specifically an external signaling mechanism to indicate, via visual, auditory or network-communicated means, inflation status of the in-dwelling balloon is depicted 14 as a unit in the context of its implementation via connection to a replacement gastrostomy tube.

### Separable Tip

The assembly/kit of the present invention further comprises a separable tip. Turning to FIG. 3, an optional, separable tip comprised of angles and shapes with taper greater, and a distal area smaller, than provided by previously disclosed tube designs is depicted. An outline of the distal end 19 of a sample tube segment 16 from replacement gastrostomy tubes of customary design, including but not limited to those including the other unique aspects of the present invention, is depicted for context. The replacement gastrostomy tubes may or may not incorporate the unique features depicted in the other Figures herein, and may be of varying materials and dimensions. The tubes may or may not include a taper as part of their design. An example shape of the separable tip of the present invention is indicated 15 17 18.

A degree of taper which exceeds that found in previously disclosed designs is depicted by gradually converging boundary lines 17 which depict an example of the taper from the wider extent at the proximal end of the tip 18 to a narrower distal end point 15 supported by the present separable tip, however the depicted angle of taper is an example and any angle or and shape that creates a converging form between the proximal and distal portions of the separable tip is covered by the present invention. The separable tip may be constructed of materials dissolvable in digestive fluid (e.g. gelatin) and/or of a size and shape such as to be excreted from a body upon separation from the replacement gastrostomy tube once placed in an in-dwelling position. In this embodiment, the actual concluding distal tip is depicted as a rounded, closed tip 15, however any terminating shape for the distal tip that is of smaller area that those provided by previously disclosed designs is within the scope of the present invention. The concluding shapes forming the tip may include any form of tapered shape or shape with a smaller distal end-point than previously disclosed gastrostomy tube distal openings, and open or closed tip designs, each including a distal end point consisting of an opening smaller than the smallest of previously disclosed gastrostomy tube distal openings. A portion of the overall tip structure 18 will be embedded within or around a replacement gastrostomy tube to secure it during the placement procedure. A surface as depicted 18 will secure, via means such as compressive friction or weak adhesive, the present invention of FIG. 3 within a distal end of a replacement gastrostomy tube until such time as a tube is placed in its in-dwelling positon and the temporary tip may be dislodged by various means to reveal the distal opening of the tube 19 for ongoing use. While the embodiment depicted discloses a tip that is manufactured as a separate unit distinct from a distal end of a replacement gastrostomy tube, other embodiments of the present invention could include a dissolvable tip constructed of a digest-able substance which is generally recognized as safe for entry to a digestive tract, which can be manufactured as a unit with a replacement gastrostomy tube, or as a separately manufactured addition to such tubes, and which may dissolve when in contact with digestive compounds and/or may be cleared by a mechanism such as a rod-and-swab type of device inserted through a gastrostomy tube to dislodge and remove the presently disclosed tip. Additionally the presently disclosed tip may be dislodged into a digestive tract and excreted, and may be dislodged by the force of incoming food products or other matter flowing from an external feeding port to a distal opening of such tubes. For safe excretion, the tip must be made of bio-compatible materials such as found on cameras or drug-delivery mechanisms which may pass through the entire intestinal tract. It may also be made of substances that can be expected to dissolve in the digestive tract (e.g. gelatin).

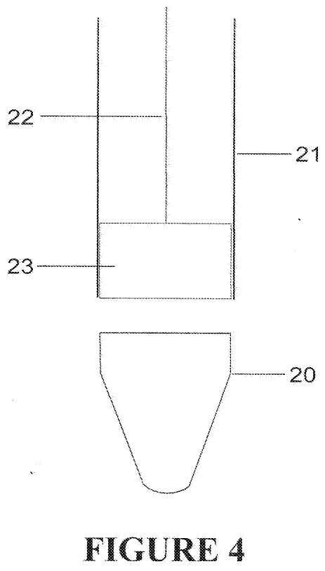

FIG. 4 depicts an example of the presently disclosed dissolvable tip 20 in the context of an example device to dislodge the temporary tip, in this example a rod 22 and swab 23 of customary design and appropriate size for a gastrostomy tube, which when temporarily inserted through the complete length of a gastrostomy tube 21 which includes the presently disclosed separable tip, would dislodge the tip through the distal opening into a digestive tract. Such a rod-and-swab is not part of the present invention and is shown only as an example of a way to dislodge the temporary tip. This procedure would be expected to occur after a tube is in its in-dwelling position such that the placement-centering action provided by the present invention would no longer be required. In this embodiment the tip 20 does not include the ridge feature depicted in FIG. 3 where the portion of the temporary tip internal to a gastrostomy tube 18 is slightly smaller than the initial external diameter of the tip, but may include such a ridge in other examples of the invention.

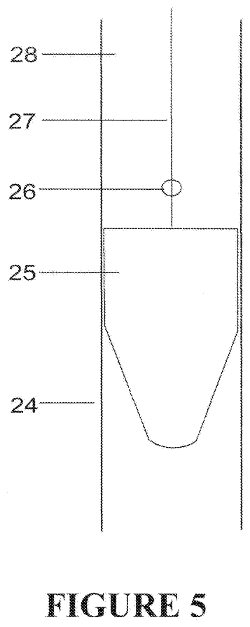

FIG. 5 depicts the temporary distal tip 25 shown in the context of being removed through a gastrostomy tube, moving in the direction from an end distal opening toward an external proximal opening outside a body, after a replacement gastrostomy tube has been has been placed in its in-dwelling location and the self-centering function provided by the tapered temporary distal tip 25 is no longer needed. In the example embodiment of FIG. 5, a flexible attachment 27 (e.g. a rod-like or string-like structure that is able to attach to the tip's proximal surface or related attachment point) is connected to an attachment point 26 opposite the distal end of the temporary tip such that the tip may be withdrawn from a tube 24 through a lumen area 28 of the tube in the direction of an external feeding port or other opening where the temporary tip 25 will exit the tube. Although an attachment point may be provided 26, the present invention covers any form of attachment between a connecting material 27 and the separable tip 25.

Materials which satisfy the requirement of having sufficient rigidity to serve the self-centering placement function and to safely enter the digestive tract are readily apparent to those familiar with the field of practice. A commonplace example found in most domestic settings is the use of a gelatin container of a size and shape sufficient to swallow (commonly referred to as “gel caps”) which contain a substance to be delivered to the digestive tract. In one example, an outer gelatin shell is dissolved in digestive fluids and contents of the “gel-cap” are deposited in a digestive tract. A similar principle would apply in the case where the presently disclosed distal tip is constructed of a material with identical or similar characteristics to gelatin or any other material which can safely be deposited into the digestive tract where it may either partially or fully dissolve, or be excreted partially or fully intact.

Devices are also well-known in the field of the art which are designed to pass completely through the digestive tract (for example, diagnostic cameras), and similar principles may be applied to removal of a dislodged temporary tip of the present invention from a gastrostomy tube once positioned in place for a tube's ongoing function.

### Mesh Reinforcement for Gastrostomy Tubes

FIGS. 6A and 6B depict two views of a mesh-latticework reinforcement 30 which may be embedded within some or all of the length of a feeding tube 29 and in some or all of adjunct tube extensions/ports as a form of structural reinforcement to increase the functional life of a tube by reducing the potential for a tube to deform from its initially constructed shape. The mesh structure may be concentrated in the boundary of the internal lumen of such tubes as depicted in the present embodiments, embedded within a wall of the tubes or predominantly within the external wall 29 of the tubes, or any combination of the above. The present embodiment shows mesh within an inner wall of the depicted tube, however this does not limit the placement of the mesh reinforcement for purposed of this disclosure. The mesh reinforcement provides a degree of additional structural rigidity beyond that provided by customary gastrostomy tubes manufactured using a predominantly constant density of tube material. The mesh used may be of any latticework dimensions suitable to provide structural improvements over solid material designs and as such the grid shape and size depicted is an example only and not a restriction on the scope of the present invention. Example materials used may include polyurethane-based formulations as described in the prior art, however the present invention is not limited to specific materials and may also include compounds with significant rigidity such as metallic compounds.

### pH Sensor for Gastrostomy Tubes

FIGS. 7A and 7B depict a ph-sensitive material 31, 32 attached to or embedded in an interior surface of a replacement gastrostomy tube such that the material will register a close approximation of the ph of fluid withdrawn from the location of such a tube's in-dwelling distal tip, and the material is, when the fluid is deposited on the material 31 32, visible to a medical practitioner. The material will have precision sufficient to assess if a distal tip is in contact with stomach acid based on the color registered on the ph-sensitive material. The ph sensitive material may be viewed through the walls of a tube or withdrawn from a tube for viewing. The tube depicted in FIGS. 7A and 7B includes the mesh reinforcement feature of FIGS. 6A and 6B however the invention of FIGS. 7A and 7B may be incorporated in any form of replacement gastrostomy tube and the mesh feature is only included in this depiction as an example and is not a requirement of the invention of FIGS. 7A and 7B. The ph-sensitive material 31 32 may be of any length, shape, size, composition and coverage area sufficient to allow a practitioner to place fluid from an in-dwelling distal tip in contact with the material. This is expected to cover a length of at least 0.5 inches and to cover at least one-quarter of the diameter of a tube, however larger or smaller coverage areas are within the scope of the currently disclosed invention. While the depiction of FIG. 7B shows the ph-sensitive material 32 as partially detached from the inner surface of the tube, any degree of partial or complete attachment, flush or otherwise, is covered by the present invention.

Having described the invention I claim:

1. An assembly for use in Replacement Gastrostomy Tube therapy, comprising one or more of:
a. an Replacement Gastrostomy Tube status device comprising a sensing module able to trigger an alarm that indicates the under inflation status of a retention balloon;
b. a temporary distal “tip” structure able to assist with the proper placement/centering of a Replacement Gastrostomy Tube in vivo;
c. a gastrostomy tube comprising,
1) a mesh latticework structure to increase the structural integrity of the Replacement Gastrostomy Tube; and/or
2) a pH sensor able to measure the acidity of fluid at distal tube opening.

2. The assembly of claim 1, wherein the Replacement Gastrostomy Tube status device comprises from end-to-end,
a. a pressure sensing unit 5 that receives electrical input from a distal retention balloon fluid pressure sensor, wherein said sensing unit is able trigger an alarm when a fluid pressure passes a threshold level;
b. a battery 6 able to power the Replacement Gastrostomy status device, optionally including any form of indication signaling a low-battery status;
c. an electrical circuit 7 comprising a processor, a memory, and a wired or wireless transmitter able to receive input from sensing unit 5, and able to compute when the fluid pressure threshold level is reached;
d. an alarm unit able to trigger an alarm when the fluid pressure threshold level is reached, said alarm being optionally configurable by a user for alarm type and, when applicable, alarm volume.

3. The assembly of claim 2, wherein the sensing unit further comprises electrical circuitry to wirelessly transmit the alarm, and/or the current Replacement Gastrostomy Tube fluid pressure level, and/or a history of the Replacement Gastrostomy Tube fluid pressure level to a remote computer, wherein said computer comprises one or more of: a remote server, and a user electronic computing device.

4. The assembly of claim 1, wherein the alarm further comprises a local auditory signal with optionally configurable volume, and/or a visual signal detectable by the user.

5. The assembly of claim 1, wherein the temporary, separable distal tip is constructed in a shape, size and material which can be mechanically, forcibly and/or chemically dislodged from a replacement gastrostomy tube in vivo, either through a distal or external (proximal) tube opening

6. The assembly of claim 5, wherein the distal tip comprises biocompatible, biodegradable material sand a shape that is able to dissolve safely in a patient's digestive system when the tip is dislodged from the tube into the patient's stomach.

7. The assembly of claim 5, wherein the tip is constructed in a shape, size and material which can, based on common principles of the art, be safely withdrawn through a distal opening in the replacement gastrostomy tube via pulling the tip out of the tube using a mechanical attachment.

8. The assembly of claim 7, wherein the mechanical attachment comprises a flexible, rod-like structure affixed to the rear-side of the tip, and able to push and pull the tip within the tube.

9. The assemble of claim 1, wherein the gastrostomy tube is comprised including a mesh-latticework structure embedded along the replacement gastrostomy tube of customary design as referenced in prior art having a main feeding tube incorporating a tubular lattice-work layer at any point on or between the interior lumen of the tube and the exterior boundary of the circumference of the tube, and extending any of the length or ports of the tube, such that commonly found flexibility of the tube is substantially retained while creating enhanced resistance to deformation from an original shape and circumference of the tube.

3. 10. The assembly of claim 9, wherein the mesh latticework structure is constructed of any material sufficient to create increased structural integrity to resist deformation of the replacement gastrostomy tubes.

4. 11. The assembly of claim 1, wherein the gastrostomy tube comprises a pH-sensitive material within the interior lumen of a replacement gastrostomy tube comprising translucent tube walls, wherein the pH sensitive material and a change in a color of the material, is visible through the translucent tube walls.

12. A method of using as assembly in a Replacement Gastrostomy Tube therapy, comprising
a. providing an assembly comprising one or more of:
a Replacement Gastrostomy Tube status device comprising a sensing module able to trigger an alarm that indicates the under inflation status of a retention balloon;
a temporary distal “tip” structure able to indicate the proper centering of the Replacement Gastrostomy Tube in vivo;
a gastrostomy tube comprising,
a mesh latticework structure to increase the structural integrity of the Replacement Gastrostomy Tube; and/or
a pH sensor material able to measure the acidity of fluid at a distal tube opening;
b. Inserting the assembly into a Replacement Gastrostomy Tube during preparation for insertion into a body of such a tube
c. Detecting by the clinician, one or more of:
an alarm for when the retention balloon is underinflated due to loss of fluid;
the distal tip is in position on the distal Replacement Gastrostomy Tube end, and the Replacement Gastrostomy Tube is properly centered during insertion;
a pH level of the material after inserting the Replacement Gastrostomy Tube into a patient's body.
